(19) United States
(12) Patent Application Publication
(10) Pub. No.: US 2008/0312507 A1
(43) Pub. Date: Dec. 18, 2008

(54) Apparatus and method for imaging-assisted intubation using pre-existing practitioner skill set

(76) Inventor: Taehoon Kim, Brentwood, CA (US)

Correspondence Address: Taehoon Kim, 304 Foothill Drive, Brentwood, CA, 94513, US

(21) Appl. No.: 11/818,831

(22) Filed: Jun. 16, 2007

(51) Int. Cl. A61B 1/267

(52) U.S. Cl. 600/188

(57) ABSTRACT

An apparatus facilitates indirect visualization of vocal cords during intubation. In one embodiment, the apparatus includes: a laryngoscope, the laryngoscope configured to be held during use by a first hand of a medical worker having two hands; a visual display device, the visual display device configured to be coupled to the laryngoscope for physical support of the visual display device during use; and an image acquisition device for acquiring imagery for display on the visual display device, a distal end of the image acquisition device configured to be disposed near a distal end of an elongated element, the elongated element configured to be physically moved during use primarily by a second hand of the medical worker, and thereby the distal end of the image acquisition device being configured to be physically moved during use primarily by the second hand, the first and second hands being the two hands of the medical worker, the imagery for helping to visualize, during use, an opening that is a target for insertion of the ET tube.

### BACKGROUND

In hospital settings or non-hospital settings, intubation of a patient may be required. For example, intubation may be required to insert an endotracheal tube (“ET tube”) into a patient's airway in order to connect an external ventilator for oxygenation. The traditional method of airway intubation includes direct visualization (i.e., seeing) of the larynx including vocal cords. In seeking direct visualization, the practitioner begins by lifting the patient's jaw and tongue base with a laryngoscope and manipulating the head and cervical portion of the neck to position the patient's larynx. This lifting of the soft tissues of the oropharynx (or, back of the throat) and/or epiglottis usually suffices to provide direct visualization of the vocal cords through the oral cavity. The distal tip of the laryngoscope generally includes a light emitter that hopefully illuminates the vocal cords. (In the present document, “distal” and “proximal” are used in reference to the medical practitioner, unless context otherwise indicates.)

However, there are many situations in which a lifting of the soft tissues in the oropharynx with a laryngoscope does not provide sufficient visualization of the vocal cords. This would be an example of a difficult airway intubation. The problem typically lies in an inability to see around soft tissues that obstruct the needed view, e.g., including but not limited to some of the following: large tonsils or base of tongue, floppy epiglottis, low lying larynx, retrognathic mandible, excessive soft tissues of the oropharynx and hypopharynx, and the like. The problem may be due to a non-paralyzed patient or to a trauma situation, in which head movement of the patient must be minimized in order not to exacerbate any injury. Lack of direct visualization of the larynx provides a dilemma for the medical practitioner performing traditional intubation because there is no way of placing the tip of the ET tube into the trachea with confidence. The tip of the ET tube may end up erroneously in the blind channel of the pyriform sinuses or in the esophageal inlet. Such erroneous placement is likely to injure the patient's hypopharynx and laryngeal structures and will lose precious time for oxygen ventilation, with perhaps fatal consequences.

The traditional method of intubating includes having the practitioner stand or kneel at the head of the bed or floor, behind the patient's supine head. As is shown in FIG. 1, typically, for a right handed practitioner 110, the laryngoscope 120 is held by the left hand 122 and the ET tube 130 is held in the right hand 132, as shown in FIG. 1. Optionally, a formable stylet (not shown) may be temporarily inserted within the ET tube. The formable stylet would temporarily lend increased rigidity to the ET tube, to facilitate more dextrous handling of the ET tube during intubation. The formable stylet may be a copper rod or tube that has some rigidity but also has enough flexibility to be hand-bendable into a desired shape by the practitioner.

In the traditional intubation method, the practitioner places the rigid laryngoscope to retract the base of tongue at the vallecula or the epiglottis 140. This action typically lifts the epiglottis out of the way, and then the vocal cords can be directly visualized. In the event that the vocal cords cannot be visualized directly, the practitioner would immediately realize that there is a difficult airway, and the practitioner would choose one or more alternative intubation methods or alternative equipment, based on the availability of time and the availability of alternative equipment. In some situations, a blind intubation (i.e., intubation without visualization of the larynx) is the only available choice. A blind intubation typically includes removal of the laryngoscope or repositioning of the head and neck and may involve use of other equipment, if there is enough time, to help in the intubating process. As mentioned above, a blind intubation can cause trauma to the patient's structures and may result in erroneous placement or lost time for ventilation.

Alternative intubation equipment and techniques have been proposed or used, for difficult intubations. In particular, alternative equipment and techniques seek to provide illumination and indirect visualization of the larynx in specific ways via cameras or fiber-optic cables.

Such existing alternative equipment and techniques that are intended to provide indirect visualization for difficult intubations generally fall into three categories.

In the first category, a camera or a distal end of a fiber-optic cable is coupled near to the distal end of an ET tube to produce an image for indirect visualization by the practitioner, e.g., on an eyepiece that must inconveniently move along with the ET tube or on a display, e.g., an external video monitor.

Examples of this first category are found in U.S. Pat. Nos. 6,929,600, 6,629,924, 5,842,973, 5,676,635, 5,607,386, 5,363,838, 5,329,940, 5,285,778, 4,742,819, 3,677,262. Examples from this category generally suffer from at least one of being difficult to use or requiring the presence of certain unwieldy equipment, e.g., certain equipment of a type not normally in-hand during traditional intubation.

In the second category of existing alternative equipment and techniques, a camera or a distal end of a fiber-optic cable is coupled near to the distal end of a laryngoscope to produce an image for indirect visualization by the practitioner. Examples of this second category are found in U.S. Pat. Nos. 6,890,298, 6,840,903, 6,354,993, 5,827,178, 4,337,761, 4,086,919. A problem with examples from this category is that the distal end of the laryngoscope lacks mobility, and therefore the camera or fiber-optic cable might not be optimally placed and might not actually succeed in providing indirect visualization.

In the third category, a single combination-type device is employed that include a laryngoscope-like portion connected to a guide that is to be in slideable contact with the ET tube during use. The guide substantially restricts movement of the distal end of the ET tube relative to the laryngoscope-like portion of the combination-type device during intubation. For example, after being restricted, the permitted movement may be substantially merely distal and proximal movement along a path defined by the guide. In one example, the guide is configured to define a lumen through which the ET tube slides. (See U.S. Pat. No. 4,337,761.) In another example, the guide is configured as a stylet around which the ET tube slides; the stylet is fixedly connected at its proximal end to the laryngoscope-like portion. (See U.S. Pat. No. 5,665,052.) In the mentioned examples, a fiber-optic cable is near the distal end of the laryngoscope-like portion of the single combination-type device to produce an image for indirect visualization by the practitioner. A problem with examples from this category includes substantial departure from the traditional method of intubation and therefore requirement for substantial training of practitioners.

### SUMMARY OF THE INVENTION

What is needed is an apparatus and a method to facilitate indirect visualization during intubation that avoid problems associated with conventional intubation apparatuses, and methods, that use direct or indirect visualization.

In an embodiment of the present invention, there is an apparatus for facilitating medical endotracheal intubation, in which an endotracheal tube, hereinafter referred to as the ET tube, is inserted via a patient's oral or nasal cavity. The apparatus includes: a laryngoscope, the laryngoscope configured to be held during use by a first hand of a medical worker having two hands; a visual display device, the visual display device configured to be coupled to the laryngoscope for physical support of the visual display device during use; and an image acquisition device for acquiring imagery for display on the visual display device, a distal end of the image acquisition device configured to be disposed near a distal end of an elongated element, the elongated element configured to be physically moved during use primarily by a second hand of the medical worker, and thereby the distal end of the image acquisition device being configured to be physically moved during use primarily by the second hand, the first and second hands being the two hands of the medical worker, the imagery for helping to visualize, during use, an opening that is a target for insertion of the ET tube.

In an embodiment of the present invention, there is an apparatus for facilitating medical endotracheal intubation, in which an endotracheal tube, hereinafter referred to as the ET tube is inserted via a patient's oral or nasal cavity. The apparatus comprises a visual display device, the visual display device configured to be coupled to a laryngoscope for physical support of the visual display device during use, the laryngoscope configured to be held during use by a first hand of a medical worker having two hands; and a display-side portion of an imagery input pathway for the visual display device, the display-side portion of the imagery input pathway configured to be capable of receiving image information from an image acquisition device that is configured to be physically moved during use primarily by a second hand of the medical worker, the first and second hands being the two hands of the medical worker.

In an embodiment of the present invention, there is an apparatus for facilitating medical endotracheal intubation, in which an endotracheal tube, hereinafter referred to as the ET tube is inserted through a patient's oral or nasal cavity, the apparatus includes: an image acquisition device for acquiring imagery for indirect visualization of vocal cords and surrounding structures during intubation; and a physical support for the image acquisition device, the physical support configured to dispose a distal end of the image acquisition device near a distal end of the ET tube during intubation, wherein the distal end of the image acquisition device moves substantially in lockstep with movement of the distal end of the ET tube by a medical worker during intubation. The apparatus is configured to be without an emitter of light, and wherein illumination for image capture is provided not by any emitter of light that moves substantially in lockstep with movement of the distal end of the ET tube by a medical worker during intubation.

### BRIEF DESCRIPTION OF THE DRAWINGS

In order to more fully describe some embodiments of the present invention, reference is made to the accompanying drawings. These drawings are not to be considered limitations in the scope of the invention, but are merely illustrative.

FIG. 1 is a schematic diagram showing a laryngoscope being held in a left hand and an ET tube being held in a right hand of the practitioner, according to the traditional airway intubation method that hopes to employ direct visualization.

FIGS. 2A-2B are schematic diagrams showing an example of a visual display that is physically supported by a laryngoscope, according to an embodiment of the present invention.

FIGS. 2C-2D are schematic diagrams showing example wired and wireless versions of the apparatus of FIGS. 2A-2B.

FIG. 3A is a schematic diagram separately showing an ET tube and, according to an embodiment of the present invention, an example of an elongated element with an image acquisition device disposed near the distal tip of the elongated element and an example of a condom for separating the elongated element from the patient.

FIG. 3B is a schematic diagram showing the ET tube, condom, and elongated element of FIG. 3A in an example arrangement suitable for intubation use.

FIG. 4 is a schematic diagram showing a zoomed-in view of the distal portion of the arrangement of FIG. 3B.

FIG. 5 is a schematic diagram showing a section view, of the arrangement of FIGS. 4 and 3B, that includes the ET tube.

FIG. 6 is a schematic diagram showing a section view, of the arrangement of FIGS. 4 and 3B, that includes the image acquisition device and does not include the ET tube.

### DETAILED DESCRIPTION OF SPECIFIC EMBODIMENTS

The description above and below and the drawings of the present document refer to examples of currently preferred embodiments of the present invention and also describe some exemplary optional features and/or alternative embodiments. It will be understood that the embodiments referred to are for the purpose of illustration and are not intended to limit the invention specifically to those embodiments. For example, preferred features are, in general, not to be interpreted as necessary features. On the contrary, the invention is intended to cover alternatives, variations, modifications and equivalents and anything that is included within the spirit and scope of the invention.

Some embodiments of the present invention seek to avoid at least some of the deficiencies of the existing alternative equipment and techniques. The deficiencies, for example, may include but are not limited to one or more of: (1) requiring the presence of certain unwieldy equipment of a type not normally in-hand during intubation, (2) requiring dramatic departure from the traditional intubation method and therefore requiring extensive training, or (3) too frequently failing to provide adequate indirect visualization.

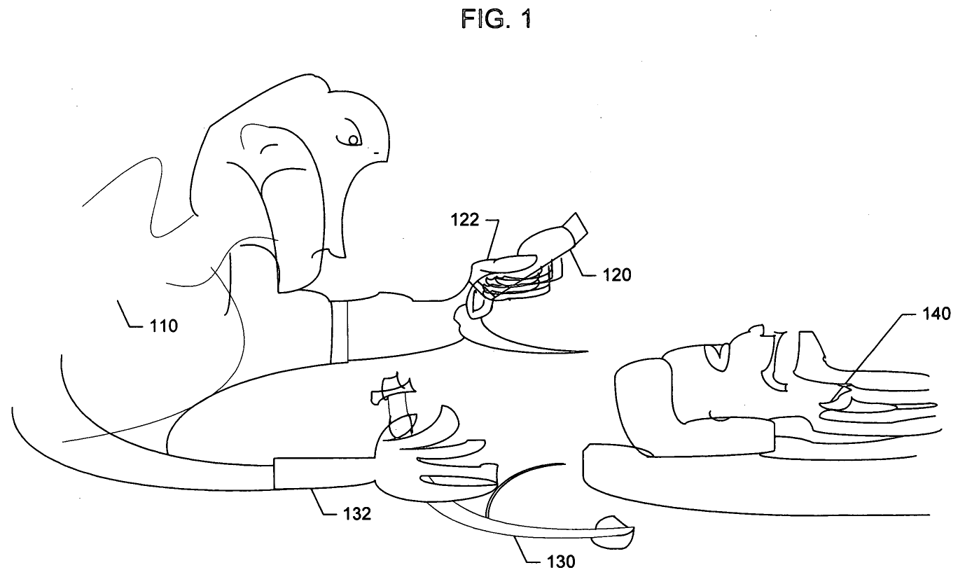

FIG. 1 is a schematic diagram showing a laryngoscope being held in a left hand and an ET tube being held in a right hand of the practitioner, according to the traditional airway intubation method that hopes to employ direct visualization. (Of course, for a left-handed practitioner, the discussion of the present document would still apply, with left and right uniformly interchanged.)

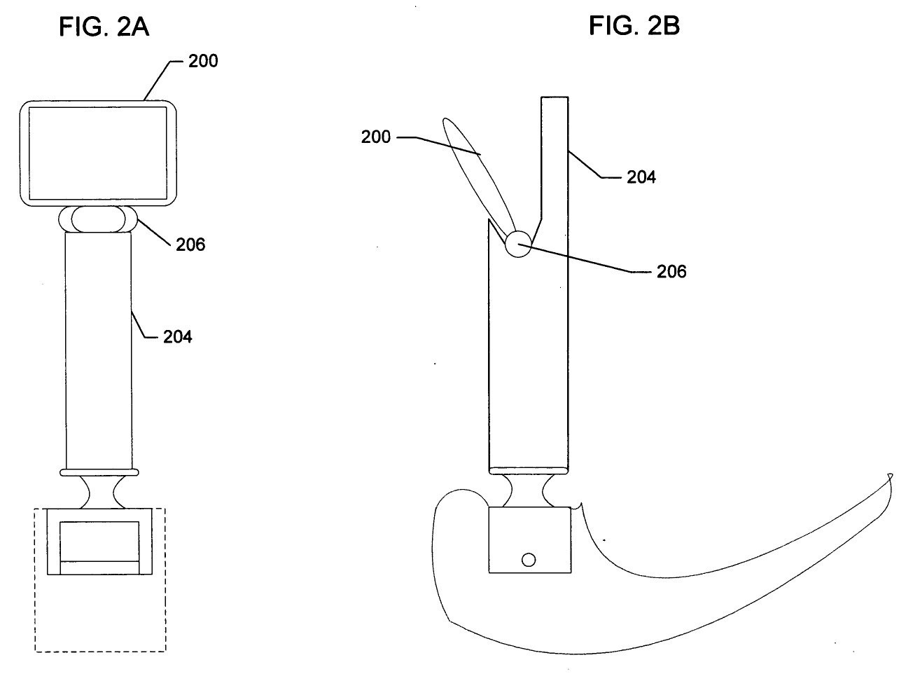

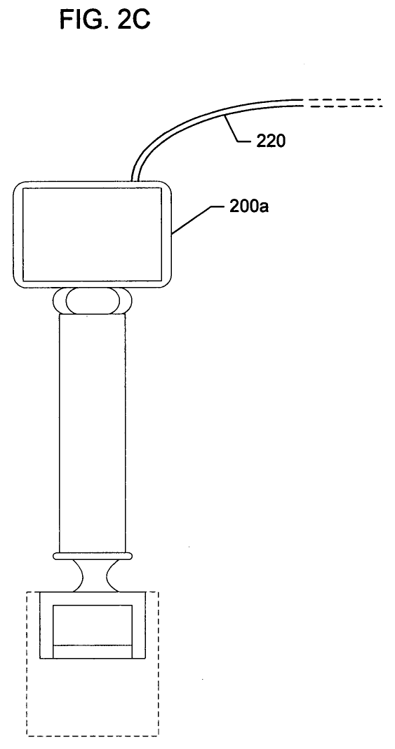

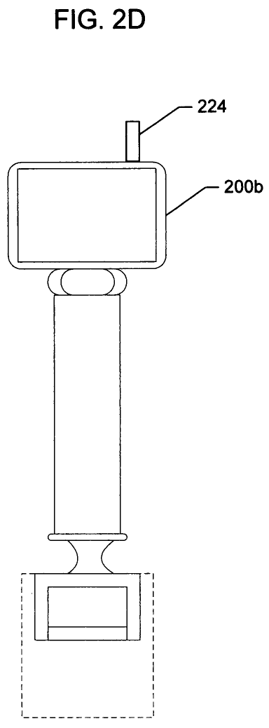

FIGS. 2A-2B are schematic diagrams showing a front view and a side view, respectively, of an example visual display 200 that is physically supported by an example laryngoscope 204, according to an embodiment of the present invention. The visual display 200 may be coupled to the laryngoscope 204 via an orientation-adjustable connector 206, e.g., a single-degree-of-freedom (pitch) hinge, or a two-degrees-of-freedom (pitch and yaw) swivel hinge, or the like. The connector 206 may be of a type that is hand-detachable (e.g., detachable without loose tools) and hand-attachable from the laryngoscope 204 via any effective type of connector, e.g., any effective snap-in or clip-on or snug-fit or clamp-on or hand-screw-on connector, or the like. Alternatively, the connector 206 may be factory-attached to the laryngoscope 204 such that the visual display 200 is not hand-detachable (e.g., not detachable without loose tools) by the user. The laryngoscope 204 may be of any effective type. For example, it may have a detachable blade and a light emitter near its distal tip and a main battery in its handle. The light emitter preferably emits visible light. In one embodiment, the light emitter may also or instead emit electromagnetic radiation in the non-visible range, e.g., infrared light. In some embodiments, the laryngoscope 204 may be considered to include the visual display 200. In some embodiments, the laryngoscope 204 may be factory configured specifically to engage, in a hand-detachable manner, the connector 206. In some embodiments, the laryngoscope 204 may be a traditional laryngoscope built without any visual display 200 in mind, and the connector 206 may be configured to retrofit onto the traditional laryngoscope, e.g., via a clamp-on band around a tubular handle, or via a snug-fit elastic cup that cups the top end of a tubular handle, or the like.

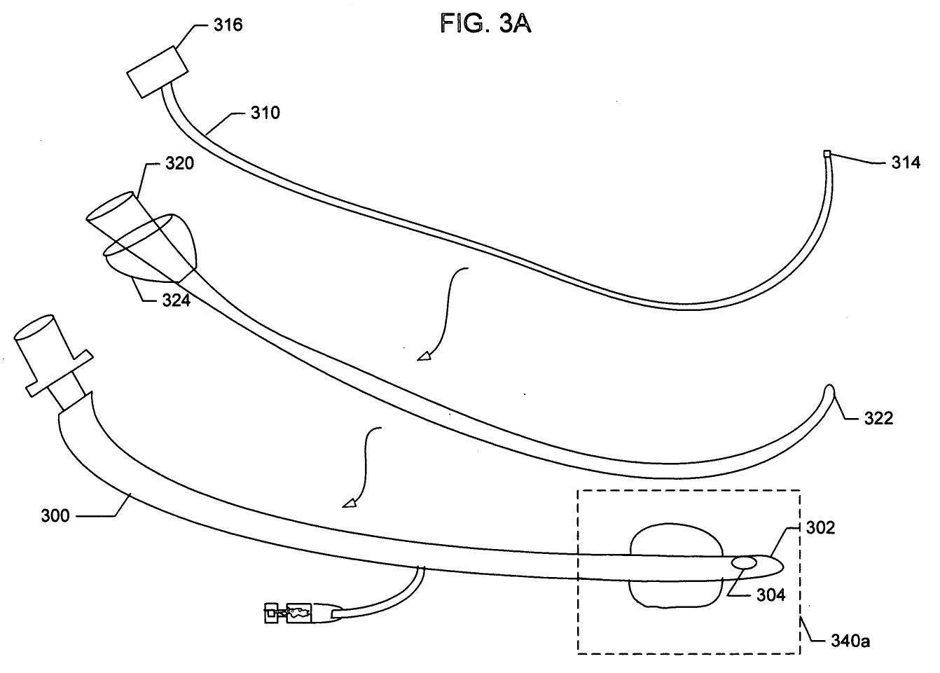

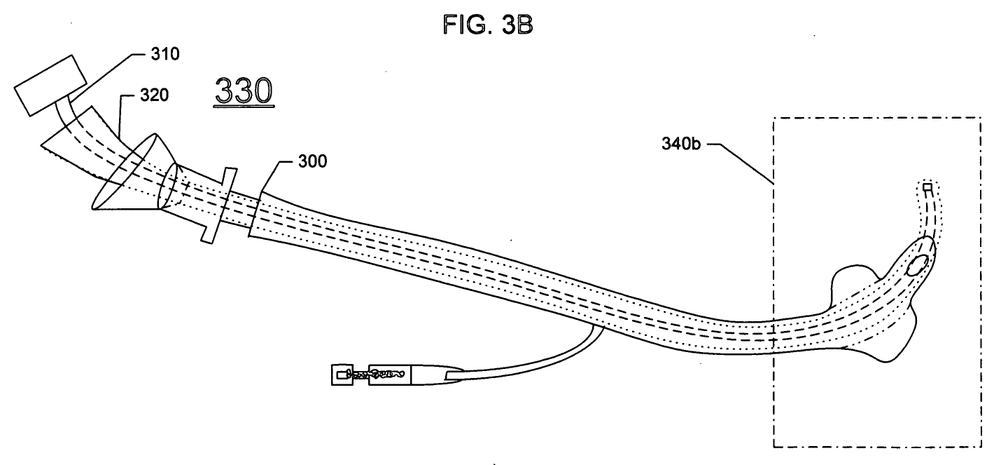

FIGS. 2C-2D are schematic diagrams showing example wired and wireless versions 200a and 200b respectively of the apparatus of FIGS. 2A-2B. In FIG. 2C, a proximal end (relative to the visual display 200) of a wire bundle 220 is shown. The wire bundle 220 conveys visual information into the visual display 200a for display. Preferably, the wire bundle 220 is in the form of a cable with a detachable multi-contact plug connector at one end for plugging into a compatible socket (not shown) on the visual display 200 unit. At the other end of the cable, the cable may be hard-wired (or, optionally, detachably plugged) to an apparatus (e.g., the apparatus of FIG. 3A) that contains an image acquisition device. In FIG. 2D, an antenna 224 is shown. The antenna 224 receives visual information into the visual display 200b for display. There is an imagery input pathway that extends from an image capture device to the visual display 200a. That portion of the pathway that is physically situated near the visual display 200a and is physically supported by the hand that physically supports the visual display 200a may be termed the display-side of the pathway. The proximal end of the wire bundle 220 or the antenna 224 is connected to the display-side of the pathway, and, when connected, may be considered to be an element within the display-side portion of the imagery input pathway. The display-side of the pathway includes any effective communication processor and associated hardware and software needed to receive imagery information for display.

The visual display 200 includes associated componentry including, for example, at least some of the following: control processor; visual processor; user controls (not specifically shown), either physical (e.g., physical buttons or switches) or virtual (touch-screen) or the like to control brightness, contrast, color, or the like; memory; control software; signal and power connectors. Optionally, the control software implements a record function and, optionally, a playback function as well. Recorded videos may be downloaded to other information devices via any effective technology, e.g., universal serial bus (USB) or the like. The record function may be useful for instructional and quality control purposes.

FIG. 3A is a schematic diagram separately showing an ET tube 300, and, according to an embodiment of the present invention, an example elongated element 310 and an example sterile condom 320 for separating the elongated element 310 from the patient. A region of interest 340a, to be discussed below, is shown by dashed lines.

The ET tube 300 is of a conventional type and need not be discussed in detail. The distal end 302 of the ET tube is an open tube-end, and, per conventional design, there is a hole 304 in the sidewall of the ET tube.

The elongated element 310 includes an image acquisition device 314 disposed near the distal tip of the elongated element 310. The image acquisition device 314 is preferably a digital video image capture device, for example, a camera-on-a-chip device (e.g., one using any effective technology, for example, charge-coupled device (CCD) or complementary metal oxide semiconductor (CMOS) technology or the like. Preferably, the image acquisition device 314 is configured to provide sufficient resolution for the intubation visualization task. The image acquisition device 314, in one embodiment, is of the type that is sensitive even to certain radiation in the non-visible range (for example, infrared light, for example, for use in combination with a laryngoscope that emits infrared light). Associated componentry, for example including power supply (e.g., battery), control circuitry and other circuitry are also included within the apparatus (or combination of apparatuses available to the medical practitioner during use) and are connected to the image acquisition device 314 using any effective configuration. For example, a battery that is included in the laryngoscope 204 shown in FIG. 2 (e.g., the main battery that also powers the laryngoscope 204's light emitter) may be used as the power supply for the image acquisition device 314, with power being conveyed via wires in the wire bundle 220; alternatively, a separate battery for powering the image acquisition device 314 may be housed within the elongated element 310, perhaps nearer its proximal end than its distal end.

Although the image acquisition device 314 is shown and described as being near the distal tip of the elongated element 310, in other embodiments only a distal end of optical fibers may be located near the distal tip of the elongated element 310, and the more proximal portions of the optical fibers convey imagery toward the medical worker's eyes, perhaps toward a digital image capture device that is not near the distal end of the elongated element 310; in such embodiments, the distal end of the optical fibers could be considered to be the image acquisition device that is disposed near the distal tip of the elongated element 310.

The elongated element 310 includes an information conveyer 316 for conveying visual information captured by the image acquisition device 314 to a visual display device, for example, the visual display 200, 200a, or 200b discussed above. The information conveyer 316 includes a wired or wireless transmitter. The information conveyer 316 or the image acquisition device 314 includes an image or video processor. The information conveyer 316 and, or including, the image or video processor may be configured to include or utilize any effective image or video or communication protocol whatsoever, digital or analog. For example, the Bluetooth digital communication protocol or the like may be used to transfer digital video of any effective format. The image conveyer may be located, as shown, near the proximal tip of the elongated element 310, or elsewhere.

The elongated element 310 preferably includes at least a substantial portion that is like a formable stylet in that it has enough flexibility to be hand-bendable into a desired shape by the practitioner and enough rigidity to retain that shape during intubation use. Optionally, the formable portion of the elongated element 310 ends short of the distal end of the elongated element 310 such that a length (e.g., about one inch or at least about one inch) at the distal end of the elongated element 310 is softer and less able to damage the patient's tissues than the formable portion of the elongated element 310 would be. For example, the length at the distal end of the elongated element 310 may be rubbery in firmness and not formable. The length at the distal end may provide functionality of a bougie if, for intubation use, it is positioned to extend beyond the distal end of the ET tube 300. (A bougie is a long, flexible, tapering or cylindrical piece of soft plastic that is inserted into a tubular passage of the body. In intubation, a bougie is sometimes pre-inserted into an ET tube such that the thin bougie is first inserted into the laryngeal inlet and then the thicker ET tube enters the inlet, guided by the bougie.) For convenience or flexibility, the image acquisition device 314 and some associated componentry may optionally be configured to be in a compact housing at the distal end of the elongated element 310, such that the housing detaches from the proximal rest of the elongated element 310; the proximal rest of the elongated element 310 may effectively be merely a conventional stylet or the like.

Preferably, the elongated element 310 does not include a light emitter (e.g., Light Emitting Diode (LED) or distal end of an optical fiber(s)) near its distal end for illuminating the patient's vocal cords for the image acquisition device 314. Rather, preferably, the image acquisition device relies merely on the light emitted by the laryngoscope and on any ambient light for illumination. Optionally, however, a light emitter could be included in the elongated element 310 near its distal tip, if additional illumination is desired.

The condom 320 is configured to accept the elongated element 310 and to prevent contamination between the elongated element 310 and the patient. The condom 320 is configured to have sufficient transparency/translucency at its most distal end 322, in order that the image acquisition device 314 can see through the distal end 322 with effective clarity for the medical practitioner to visualize body structures. For intubation use, the condom 320, with the elongated element 310 within, is configured to be first inserted into the ET tube 300.

A stopper 324 is preferably included with the condom 320 in order to fix the maximum depth of insertion of the condom 320 into the ET tube 300. Preferably, the stopper 324 is slideable along the length of the condom, in order to allow practitioner-control of the depth of insertion. In one embodiment, the stopper 324 is a tapered washer that acts as a compression washer to wedge into the proximal end of the ET tube 300 and to wedge against the condom 320 to thereby prevent mutual movement along the mutual axis between the ET tube 300 and the condom 320. In embodiments in which a stopper 324 is not included, depth of insertion would be fixed by other mechanisms, for example, friction between the condom 320 and the ET tube 300, enhanced as necessary by the grip of the medical practitioner.

The condom 320 can be of any effective condom material. For example, it may be of a flexible fluid-impermeable material. In one embodiment, the condom 320 may include the formable portion as described above in connection with the elongated element 310, and the elongated element 310 may be configured to be without the formable portion.

In an alternative embodiment, no condom 320 is used, and an embodiment of the elongated element 310 is directly inserted into the ET tube 300 to provide indirect visualization during intubation, and, optionally, to provide functionality of a formable stylet or bougie via an included formability feature as described above. Preferably, this embodiment of the elongated element 310 that is not used with a condom 320 is configured to withstand high heat such that it may be autoclaved for sterilizing. Or, this embodiment of the elongated element 310 that is not used with a condom 320 may be configured to be a disposable product. Or, the visualization-related features of the elongated element 310 may be integrated into the disposable ET-tube itself, such that the distal tip of the disposable ET-tube would include a portion that houses the image acquisition device 314; in this alternative embodiment, the enhanced ET-tube itself may be considered to be the embodied elongated element.

FIG. 3B is a schematic diagram showing the ET tube 300, condom 320, and elongated element 310 of FIG. 3A in an example arrangement 330 suitable for intubation use. A region of interest 340b, to be discussed below, is shown by dashed lines. For intubation use, the elongated element 310 is slid into the condom 320, and the combination is slid into the ET tube 300, as has been discussed above.

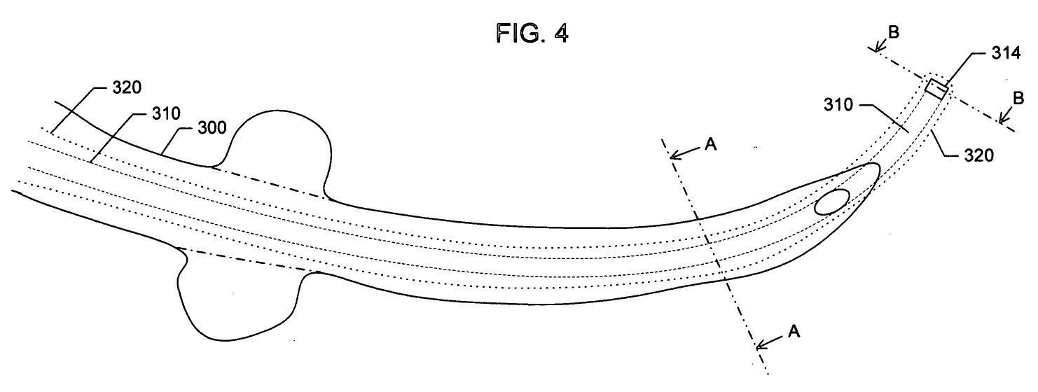

FIG. 4 is a schematic diagram showing a zoomed-in view of the distal portion of the arrangement 330 of FIG. 3B. The zoomed-in view corresponds roughly to the region 340b of FIG. 3B.

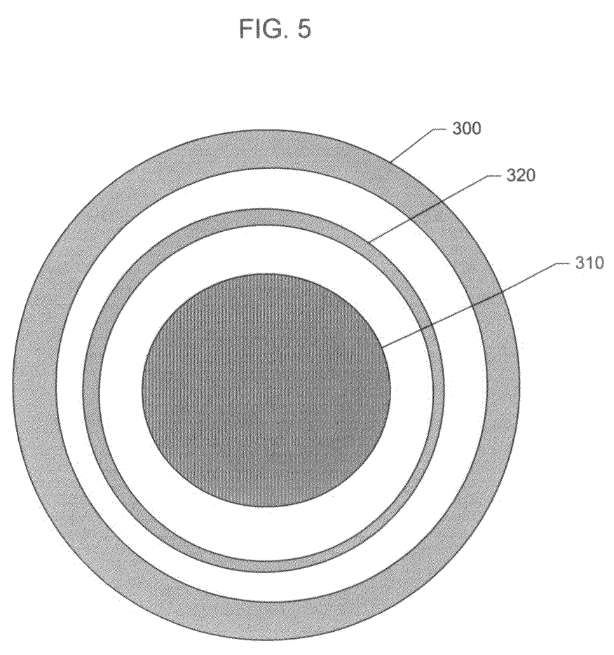

FIG. 5 is a schematic diagram showing a section view, of the arrangement 330 of FIGS. 4 and 3B, that includes the ET tube. The section view is indicated by the section line A in FIG. 4. The section view shows the ET tube 300, the condom 320, and elongated element 310.

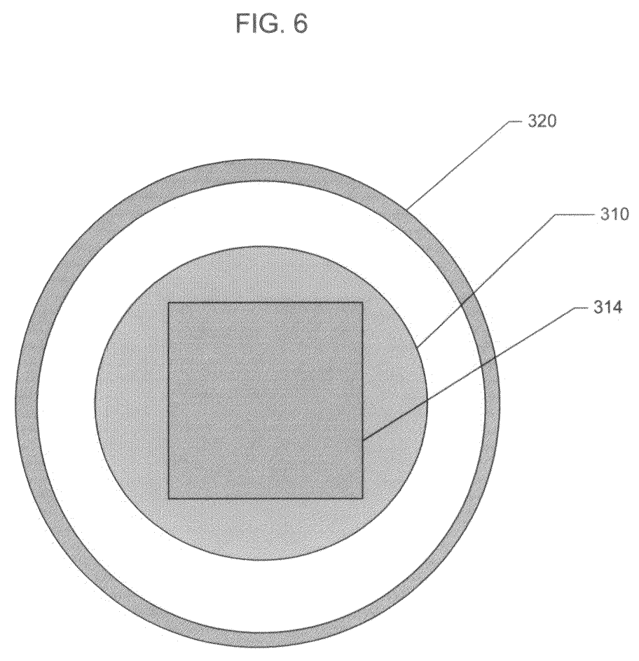

FIG. 6 is a schematic diagram showing a section view, of the arrangement 330 of FIGS. 4 and 3B, that includes the image acquisition device and does not include the ET tube. The section view is indicated by the section line B in FIG. 4. The section view shows the condom 320, the elongated element 310 and the image capture device 314.

In an alternative embodiment, the image acquisition device 314 is used to provide information not to a visual display on a laryngoscope, but to a head/helmet mounted display worn by the medical practitioner (e.g., a battlefield medic). One example of such an embodiment during use would have a laryngoscope (or elongated element 310) that emits infrared light (and, e.g., substantially no visible light) for illumination and an image acquisition device 314 that sees infrared light (for increased stealth and safety).

Throughout the description and drawings, example embodiments are given with reference to specific configurations. It will be appreciated by those of ordinary skill in the art that the present invention can be embodied in other specific forms. The scope of the present invention, for the purpose of the present patent document, is not limited merely to the specific example embodiments of the foregoing description, but rather is indicated by the appended claims. To mention just one example, although certain elements of an image-capturing and display system might be described as being located at a specific one of the left or right hand apparatuses, it should be understood that, because the left and right hand apparatuses are operatively coupled or in communication, some such elements (e.g., certain processors or processing tasks) may be allocated in either of the apparatuses, depending on designer choice. All changes that come within the meaning and range of equivalents within the claims are to be considered as being embraced within the spirit and scope of the claims.

What is claimed is:

1. An apparatus for facilitating medical endotracheal intubation, in which an endotracheal tube, hereinafter referred to as the ET tube, is inserted via a patient's oral or nasal cavity, the apparatus comprising:
a laryngoscope, the laryngoscope configured to be held during use by a first hand of a medical worker having two hands;
a visual display device, the visual display device configured to be coupled to the laryngoscope for physical support of the visual display device during use;
an image acquisition device for acquiring imagery for display on the visual display device, a distal end of the image acquisition device configured to be disposed near a distal end of an elongated element, the elongated element configured to be physically moved during use primarily by a second hand of the medical worker, and thereby the distal end of the image acquisition device being configured to be physically moved during use primarily by the second hand, the first and second hands being the two hands of the medical worker, the imagery for helping to visualize, during use, an opening that is a target for insertion of the ET tube.

2. An apparatus as described in claim 1, further comprising the elongated element wherein the elongated element includes a hand-formable length.

3. An apparatus as described in claim 2, wherein the elongated element includes a bougie at its distal end, the bougie having less rigidity than the hand-formable length of the elongated element.

4. An apparatus as described in claim 1, further comprising the elongated element, wherein the elongated element includes a bougie at its distal end, the bougie having less rigidity than the hand-formable length of the elongated element.

5. An apparatus as described in claim 1, further comprising a condom for minimizing contact between the elongated element and the patient.

6. An apparatus as described in claim 1, further comprising the elongated element, wherein the elongated element is configured to be without an emitter of light for illumination for image capture.

7. An apparatus as described in claim 1, wherein a wire bundle couples the visual display device to the image acquisition device.

8. An apparatus as described in claim 7, wherein the laryngoscope includes a light emitter and a main battery that powers the light emitter, and wherein the main battery also powers the image acquisition device.

9. An apparatus as described in claim 1, wherein the image acquisition device comprises a digital imaging chip, and the imagery includes video imagery.

10. An apparatus as described in claim 1, wherein the visual display device and the image acquisition device are configured to utilize a wireless communication channel for conveying imagery content.

11. An apparatus for facilitating medical endotracheal intubation, in which an endotracheal tube, hereinafter referred to as the ET tube is inserted through a patient's oral or nasal cavity, the apparatus comprising:
an image acquisition device for acquiring imagery for indirect visualization of vocal cords and surrounding structures during intubation; and
a physical support for the image acquisition device, the physical support configured to dispose a distal end of the image acquisition device near a distal end of the ET tube during intubation, wherein the distal end of the image acquisition device moves substantially in lockstep with movement of the distal end of the ET tube by a medical worker during intubation;
wherein the apparatus is configured to be without an emitter of light, and wherein illumination for image capture is provided not by any emitter of light that moves substantially in lockstep with movement of the distal end of the ET tube by a medical worker during intubation.

12. An apparatus for facilitating medical endotracheal intubation, in which an endotracheal tube, hereinafter referred to as the ET tube is inserted via a patient's oral or nasal cavity, the apparatus comprising:
a visual display device, the visual display device configured to be coupled to a laryngoscope for physical support of the visual display device during use, the laryngoscope configured to be held during use by a first hand of a medical worker having two hands; and
a display-side portion of an imagery input pathway for the visual display device, the display-side portion of the imagery input pathway configured to be capable of receiving image information from an image acquisition device that is configured to be physically moved during use primarily by a second hand of the medical worker, the first and second hands being the two hands of the medical worker.

13. An apparatus as described in claim 12, further including a connector configured for removably coupling the visual display by hand by a medical practitioner to the laryngoscope.

14. An apparatus as described in claim 13, wherein the connector is configured to couple to a laryngoscope without requiring a corresponding connector to have been pre-built into the laryngoscope.
